(19) United States
(12) Patent Application Publication
(10) Pub. No.: US 2025/0031630 A1
(43) Pub. Date: Jan. 30, 2025

(54) Methods And Systems For Use In Converting Thresholds In Plant Breeding Based On Stage(s)

(71) Applicant: MONSANTO TECHNOLOGY LLC, Saint Louis, MO (US)

(72) Inventors: Adam David SCOTT, Maryland Heights, MO (US); Vivek SHARMA, Sacramento, CA (US)

(21) Appl. No.: 18/784,693

(22) Filed: Jul. 25, 2024

Related U.S. Application Data
(60) Provisional application No. 63/529,333, filed on Jul. 27, 2023.

(51) Int. Cl. A01G 7/00

(52) U.S. Cl. CPC A01G 7/00

(57) ABSTRACT

Systems and methods for converting thresholds in plant breeding are provided. One example computer-implemented method includes accessing data indicative of hybrid plants at a first stage of a breeding pipeline, where the data includes values of parameter(s) of the hybrid plants; imposing a first threshold(s) included in a product definition for the breeding pipeline on the accessed data, whereby ones of the hybrid plants are considered at the first stage of the breeding pipeline based on satisfying the first threshold(s), and where the first threshold(s) is specific to one of the parameter(s); measuring the value(s) of the parameter(s) for the considered ones of the multiple hybrid plants; generating, based on an aggregate of the measured values of the parameter(s) for the considered ones of the multiple hybrid plants, a second threshold(s); and repeating the above for a second stage of the breeding pipeline with the second threshold(s).

### CROSS-REFERENCE TO RELATED APPLICATION

This application claims the benefit of, and priority to, U.S. Provisional Application No. 63/529,333, filed Jul. 27, 2023. The entire disclosure of the above application is incorporated herein by reference.

### FIELD

The present disclosure generally relates to methods and systems for use in converting thresholds in plant breeding, based on stage(s) (or generations, etc.), and in particular to methods and systems for use in converting thresholds of product definitions backwards in plant breeding stages, and executing plant breeding decisions based thereon.

### BACKGROUND

This section provides background information related to the present disclosure which is not necessarily prior art.

In developing plants, it is known to make modifications to the plants, either through selective breeding or genetic manipulation, to enhance the performance of the plants. When a desirable improvement is then achieved through development, quantities of that plant are generated for distribution. Throughout the development, decisions are made based on data related to the characteristics or traits of the plants in current generations and prior generations. Traditionally, as part of selection decisions for further development, samples are taken from the plants and tested so that plants having the desired performance are advanced. Plant development may involve a large number of possible crosses of inbreds across multiple stages, and into hybrids, from which final breeding decisions are made.

### SUMMARY

This section provides a general summary of the disclosure and is not a comprehensive disclosure of its full scope or all of its features.

Example embodiments of the present disclosure generally relate to converting thresholds in plant breeding based on stage(s).

In one example embodiment, such a method generally includes accessing, by a computing device, data indicated of multiple hybrid plants at a first stage of a breeding pipeline, the data includes values for one or more parameters of the multiple hybrids; imposing, by the computing device, one or more first-stage thresholds included in a product definition on the accessed data, whereby ones of the multiple hybrids are retained based on satisfying the one or more thresholds, each of the one or more threshold specific to one of the one or more parameters; measuring the values of the one or more parameters for the retained ones of the multiple hybrids; generating, based on an aggregate of the measured values of the one or more parameters for the retained one of the multiple hybrids, one or more second stage thresholds; repeating the above for a second stage with the one or more second stage threshold in place of the one or more first stage thresholds; and advancing one or more prior hybrid plants from the second stage to the first stage based on the second thresholds.

In another example embodiment, such a method generally includes converting one or more thresholds of a product definition backward through at least multiple hybrid stages and multiple inbred stages of the breeding pipeline for a hybrid plant, each of the one or more thresholds specific to a parameter of the hybrid plant.

Example embodiments of the present disclosure also generally relate to systems and/or non-transitory computer-readable storage media having executable instructions for converting thresholds in plant breeding, which when executed by at least one processor, cause the at least one processor to perform one or more of the operations included in the above method.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments, are not all possible implementations, and are not intended to limit the scope of the present disclosure.

FIG. 1 illustrates an example system of the present disclosure configured for converting thresholds in plant breeding pipelines, based on one or more stages thereof;

FIG. 2 is a block diagram of an example computing device that may be used in the system of FIG. 1; and

FIG. 3 illustrates a flow diagram of an example method, which may be used in (or implemented in) the system of FIG. 1, for use in converting thresholds in plant breeding between different stages of a plant breeding pipeline.

Corresponding reference numerals indicate corresponding parts throughout the several views of the drawings.

### DETAILED DESCRIPTION

Example embodiments will now be described more fully with reference to the accompanying drawings. The description and specific examples included herein are intended for purposes of illustration only and are not intended to limit the scope of the present disclosure.

Breeders often rely on a product definition in connection with breeding decisions, whereby hybrids are created and selected, for example, with the product definition in mind. Product definitions are defined by one or more parameters, whereby the product definitions may be demarked by a threshold associated with regionality, stature, yield., etc. However, as breeding often occurs over multiple stages, from inbreds to hybrids, it is not generally feasible to assess the particular inbred or hybrid (broadly, product) with respect to the product definition, especially at earlier stages, given gains and/or performance changes in the breeding pipeline over subsequent ones of the multiple stages (i.e., it is unrealistic to expect early stage inbreds/hybrids to satisfy a final product definition, whereby products outside the definition may come to satisfy the definition over stages).

Uniquely, the systems and methods herein provide for converting product definition thresholds, through one or multiple stages in a plant breeding pipeline.

In particular, product definitions for particular plants/seeds may include one or more thresholds for certain parameters. The parameters, such as, for example, regionality, stature, weight, yield, etc., are expressed in terms of the final product, and not in terms of the earlier stages of inbred/hybrids. The computing device(s) herein converts the thresholds from the end product definition backward, based on modeling historical data, into thresholds for one or more of the prior stages of the breeding pipeline (e.g., per parameter, per stage, etc.). In this manner, the thresholds are converted to the specific stage at which decisions and/or selections are to be made. This provides enhanced understanding of product definitions at earlier stages, in connection with breeding decisions, whereby the performance of the breeding pipeline and decisions therein are improved.

As such, the systems and methods herein provide a technical solution to a technical problem, whereby computer-aided breeding decisions are made based on current stage thresholds, rather than end stage thresholds. In this way, thresholds of parameter(s) is (are) defined for the end product(s) without unintentionally limiting prior stage thresholds of said parameter(s). This is contrary to conventional technical techniques to advance products from stage to stage.

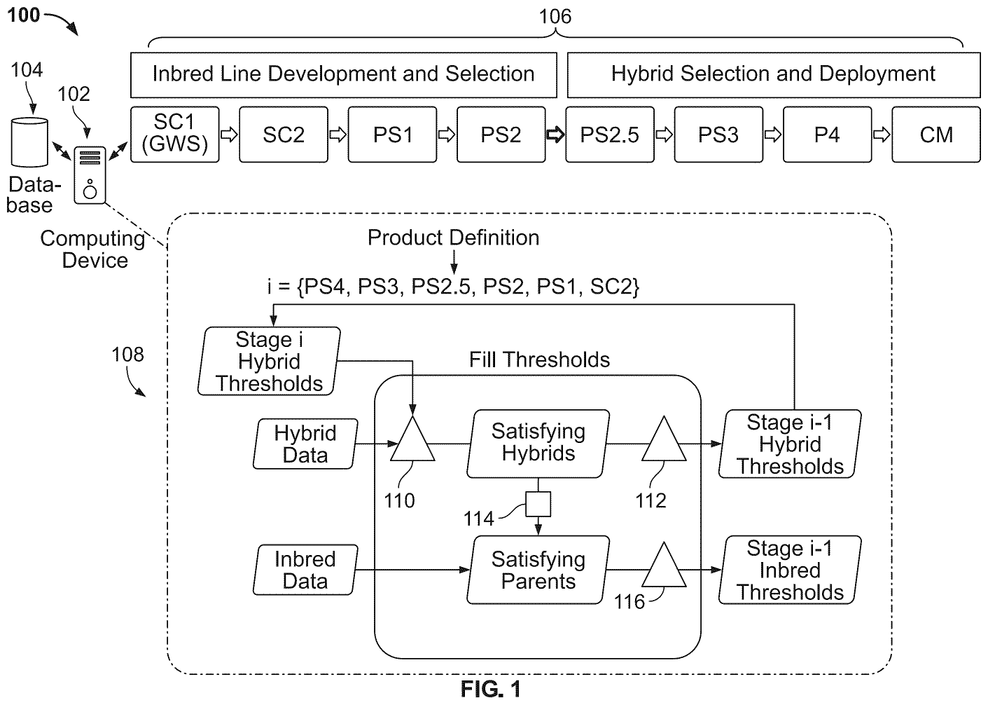

FIG. 1 illustrates an example system 100 in which one or more aspects of the present disclosure may be implemented. Although the system 100 is presented in one arrangement, other embodiments may include the parts of the system 100 (or additional parts) arranged otherwise depending on, for example, types of seeds and/or plants included in the system 100, sources of data for the seeds and/or plants, types of data (e.g., phenotypic data, geolocation data, etc.), stages of a breeding pipeline included in the system 100, etc.

In the example embodiment of FIG. 1, the system 100 generally includes a computing device 102 and a database 104. The database 104 is coupled to (and/or is otherwise in communication with) the computing device 102, as indicated by the arrowed line. The computing device 102 is illustrated as separate from the database 104 in FIG. 1, but it should be appreciated that the database 104 may be included, in whole or in part, in the computing device 102 in other system embodiments.

As shown in FIG. 1, the system 100 includes a breeding pipeline 106 (broadly, a breeding environment or breeding program), which includes various different stages for testing and advancing seeds.

In this example embodiment, the breeding pipeline 106 is described with reference to, and is generally directed to, corn. However, it should be appreciated that the methods disclosed herein are not limited to corn and may be employed in a plant breeding pipeline/program relating to other plants, for example, to improve any fruit, vegetable, grass, trees, or ornamental crops, including, but not limited to, maize (Zea mays), soybean (Glycine max), cotton (Gossypium hirsutum), peanut (Arachis hypogaea), barley (Hordeum vulgare); oats (Avena sativa); orchard grass (Dactylis glomerata); rice (Oryza sativa, including indica and japonica varieties); sorghum (Sorghum bicolor); sugar cane (Saccharum sp); tall fescue (Festuca arundinacea); turfgrass species (e.g., species: Agrostis stolonifera, Poa pratensis, Stenotaphrum secundatum, etc.); wheat (Triticum aestivum), and alfalfa (Medicago sativa), members of the genus Brassica, including broccoli, cabbage, cauliflower, canola, and rapeseed, carrot, Chinese cabbage, cucumber, dry bean, eggplant, fennel, garden beans, gourd, leck, lettuce, melon, okra, onion, pea, pepper, pumpkin, radish, spinach, squash, sweet corn, tomato, watermelon, honeymelon, cantelope and other melons, banana, castorbean, coconut, coffee, cucumber, Poplar, Southern pine, Radiata pine, Douglas Fir, Eucalyptus, apple and other tree species, orange, grapefruit, lemon, lime and other citrus, clover, linseed, olive, palm, Capsicum, Piper, and Pimenta peppers, sugarbeet, sunflower, sweetgum, tea, tobacco, and other fruit, vegetable, tuber, and root crops. The methods herein may also be used in conjunction with non-crop species, especially those used as model organisms, such as, for example, Arabidopsis.

The breeding pipeline 106 generally defines a pyramidal progression, whereby it starts with a large number of potential inbreds and narrows the inbred (or resulting hybrids) through different stages (or in some examples through different generations, etc.) to select preferred and/or desired ones thereof based on one or more product definitions. Each product definition is a definition of the desired, target or optimal commercial product based on, for example, parameter threshold(s). The breeding pipeline 106 typically involves subjecting the plants to testing, whereby additional data is determined and further selections, per stage, are made. In certain breeding pipelines (e.g., large industrial breeding pipelines, etc.), this process may involve making hundreds of thousands of selections and performing testing on hundreds, thousands, or more plants in multiple stages over several years to arrive at a potential commercial product. That is, the breeding pipeline 106 comprises many processes designed to reduce a large number of inbreds down to a relatively few number of definition specific commercial products.

With reference to FIG. 1, the stages may include any different variety of stages, based on, for example, the particular type of plants being bred (and/or seed being grown) in the breeding pipeline 106. In this example embodiment, the plants include maize or corn, whereby the inbreds may be created from conventional self-crossing or double-haploid and induction. Then, again for maize or corn, the hybrids are a combination of a female inbred and a male inbred (for example, denoted F+M). The maize or corn is bred through the multiple different stages of the breeding pipeline, with ones of the plants being removed (e.g., dropped, etc.), per stage, while others are advanced to next stages. The database 104 then includes data for each of the stages and each of the plants (e.g., along with advance/drop data, etc.) (e.g., for hundreds, thousands, tens of thousands, etc. of plants). At this point, it should be appreciated that the plants removed, per stage, are not necessarily dropped from the pipeline. They may remain in the pipeline, but not advanced to next stages in this example embodiment.

In the specific illustrated embodiment, the breeding pipeline 106 includes multiple stages, including: an origin stage, screening stages (SC1, SC2) (e.g., for testing, evaluating, etc., the seeds/plants; etc.), multiple product stages (PS1, PS2, PS2.5, PS3, PS4) (e.g., for growing, etc., the seeds/plants; etc.), and a commercial testing stage (CM). In addition, a pre-stage generally includes origin population development and selection, for example, where inbreds are developed from origin populations. The SC1 stage includes a first stage of inbred screening, and the SC2 stage includes a second stage of inbred screening. The different product stages represent testing stages for testing and selection of products across locations, regions and years. It should be noted that the stage PS2 transitions from advancing on an inbred basis only (e.g., a general combining ability (GCA) basis, etc.) to include advancing on a hybrid basis (e.g., a specific combining ability (SCA) basis, etc.), and, the CM stage represents a stage at which commercial products are tested. As indicated above, the database 104 then includes data for the plants in the different stages, over multiple cycles of the breeding pipeline 106, over various years. It should be understood that other stages or other combinations of stages may be included in other breeding pipelines, for example, depending on the particular type of plant, etc.

Each of the stages of the breeding pipeline 106 may be associated with one or more fields (e.g., in which the seeds may be planted and the plants may be grown, etc.). Each of the stages may be associated with one or more same or different fields. In this way, the breeding pipeline 106 may include dozens, hundreds, or thousands, or more or less fields, which are owned, operated and/or controlled, at least in part, by a breeder (or multiple breeders) (not shown) associated with the breeding pipeline 106. The fields may include any different type of fields (e.g., plots of land, greenhouses, growing pots or beds, etc.), of various sizes/acreages, in one or more regions, states, territories, counties, countries, etc. In connection therewith, the fields may be more broadly referenced as growing spaces for the plants.

In connection with one or more breeding operations for one or more types of plants, the fields are planted year over year (or season over season), with the same or different plants, and then harvested consistent with seasons of the plants included therein. The seeds planted in the fields, in this example embodiment, include multiple different types or varieties of seeds/plants. Each of the seeds, in turn, may include an inbred or a hybrid (i.e., a combination of lines), depending on the particular stage of the breeding pipeline 106, including multiple different varieties and/or types of seeds at multiple different stages thereof. Across the multiple fields of one or more stages, and over multiple years, the system 100 may involve hundreds, thousands, tens of thousands, hundreds of thousands or more (or less) inbreds and/or hybrids. The inbred lines in turn may provide hundreds or thousands or more distinct hybrids, i.e., one female inbred line and one male inbred line. In this example, again, the plants are corn or maize, but may be otherwise in other embodiments.

As part of the operations of the breeding pipeline 106 (and the different stages included therein), then, substantial data related to the lines, hybrids, phenotypic performance, fields, etc., is collected, organized, and stored in the database 104.

That is, in the various stages, the plants (e.g., plants, seeds, etc.) are tested for presence (or absence) of one or more traits, through one or more techniques known in the art of plant breeding. Such techniques may include any number of tests, trials, or analyses known to be useful for evaluating plant performance, including any phenotyping or genotyping assays known in the art. Common examples of seed phenotypes include size, shape, surface area, volume, mass, and/or quantity of chemicals in at least one tissue of the seed, for example, anthocyanins, proteins, lipids, carbohydrates, etc., in the embryo, endosperm or other seed tissues. Where a plant (e.g., cultivated from a seed, etc.) has been selected or otherwise modified to produce a particular chemical (e.g., a pharmaceutical, a toxin, a fragrance, etc.), the seed can be assayed to quantify the desired chemical. Examples of genetic analyses may include any form of nucleic acid detection and/or characterization, including sequencing, genotyping by sequencing, detection and characterization of sequences associated with certain alleles and/or quantitative trait loci, allele frequencies in a population of seeds, transgene, or RNA sequences in that a user is interested. With that said, it should be appreciated that the testing phase of the breeding pipeline 106 in the illustrated embodiment is not limited to certain or particular genotyping or phenotyping techniques as any method and/or technology suitable to aid in the determination of a genotype and/or phenotype of the plants at any stage of the life cycle may be used.

Regardless of the testing techniques involved, data is generated for the specific plants, and the computing device 102 is configured to store the data in the database 104. In particular, for example, the data may include various different types of data, which may represent, without limitation, parameters/characteristics of the plants (or seeds) prior to planting, at planting, during growing, and/or during/after harvest (or therebetween); characteristics of the fields, conditions of the fields and/or characteristics/conditions associated therewith before, during and/or after planting of the fields; and/or timing associated with the planting and/or harvesting of the plants; etc. The parameter data may indicate yield, car height, moisture content, green snap, plant height, stalk strength, weight, etc. It should be appreciated that more or less, or different, traits may be represented in other system embodiments. Further, the data may be indicative of each specific plant, by identifier (e.g., unique number, etc.) planted in the fields, a type of the plant (e.g., corn, etc.), a genomic description of the plant (e.g., trait stack, etc.), an identification of the parent lines (e.g., for hybrids, etc.), relative maturity (RM), etc. The data also includes a planting date of the crop in the given field, any treatments (e.g., fertilizer, herbicide, insecticide, irrigation, etc.) applied to the field, soil conditions, precipitation, solar radiation, moisture, etc. The data may also include, without limitation, performance data related to the line, such as, for example, yield, height, lodging, insect and/or drought resistance, strength, etc.

The data may also include data indicative of phenotypic traits, etc., of the lines or hybrids, which may be expressed, summarized, processed, or aggregated in one or more different manners. For example, the data indicative of phenotypic traits may be compiled into one or more best linear unbiased predictions (BLUPs) for the specific traits. In particular, yield of a line may be expressed as a BLUP, which is a linear regression or adjusted mean of the yield data based on the historical data for the inbred line (e.g., over one year, two years, or three years, etc.). Additionally, a BLUP can be obtained to indicate general combining ability of inbred parents of planted hybrids. Separately, a BLUP indicating specific combining ability of planted hybrids may inform the specific parent pair's performance comprising a given hybrid.

In this example embodiment, the data may further include an indication of the lines and/or hybrids being advanced from stage to stage in the breeding pipeline 106. The advancement of the inbred/hybrid is, for example, relative to a specific threshold. For example, the breeding pipeline 106 may include a number of stages, as illustrated in FIG. 1, for example, and the advancement of the hybrid is a 1 if the hybrid achieves a particular stage in the breeding pipeline 106 and a 0, if not. In this example, the breeding pipeline 106 includes historical data indicative of prior inbred selections and hybrid selections. In connection therewith, the inbred selection stages include SC2, PS1, PS2, and the hybrid selection stages include PS2.5, PS3, PS4, and CM, as shown in FIG. 1. The advancement of the inbreds or hybrids is then indicated in the data in the database 104. It should be appreciated that one or more similar or distinct stage thresholds may be relied upon to determine fate in other system embodiments.

The historical data in the database 104 may be organized by year (e.g., Y1, Y2, Y3 . . . . YN, etc.), or by inbred, hybrid, or field, or by location (e.g., region, territory, state, etc.). In each year, for example, the data is then organized further by crop or plant, or by region. In general, the data may be organized by region, or market, or submarket. In doing so, a region may have multiple markets, and a market may have multiple submarkets. A submarket, then, may include a particular type of product, for example, white corn, waxy corn, silage corn, etc. For example, data for the United States may include data for all of the United States together, or data for the Midwest and/or South, etc. may be separate from the data for the rest of the United States. Similarly, data for Europe may be included together or separated by region (e.g., by country, etc.). It should be appreciated that the historical data may be stored consistent with the different regions, years, markets, etc., or may be merely accessed (or filtered) consistent with a particular market, region, year, etc.

In connection with the above, a breeder associated with the breeding pipeline 106 inputs and/or specifies one or more product definitions for CM stage plants to be generated over time. The product definitions may be influenced by current product demand, environmental conditions, expected product demand, and/or other factors related to satisfying the specific demand of growers and/or resources available to produce the resulting commercial products, etc. The product definitions generally include or define parameters and a collection of thresholds related to the specific parameters of the commercial product, which are defined, generally, based on agronomics and pathology, etc. One example product definition is provided in Table 1, below. It should be appreciated that the product definitions may vary in other examples, to include one or more of the same or different parameters and/or associated thresholds, different plants, etc.

TABLE 1

| Parameter | Threshold |
|---|---|
| Adaptation | Broad acre |
| Market Opportunity | 13M acres |
| Relative Maturity (RM) | 102-108 days |
| Test Weight | −1 (BLUP) |
| Stature | 2 (BLUP) |
| Lodging | 0 (BLUP) |
| Yield | 1 (BLUP) |
| Anthracnose Stalk Rot | 4.5 (rating) |

It should be appreciated that the product definition is provided to define the commercial product, which is expected to exit from the plant breeding pipeline 106. However, decisions to create the commercial product consistent with the product definition include decisions at earlier stages, for example, PS1, PS2, PS2.5, etc., whereby the breeder, or automated selection, may not understand, properly, how to interpret the product definition thresholds at those prior stages to provide for the final commercial product from stage CM.

In this example embodiment, the computing device 102 is configured to convert the product definition, backwards through the one or more stages of the breeding pipeline 106, based on the historical data in the database 104, to provide the product definition in terms of those specific stages of the breeding pipeline 106, at which decisions are to be made.

In particular, as shown in FIG. 1, for a product definition, the computing device 102 includes an iterative conversion architecture 108, which configures the computing device 102 to convert the product definition, per stage and/or per parameter. Specifically, the initial iteration of the architecture 108 relies on the product definition of the commercial product. The computing device 102 is configured to retrieve data for available existing hybrids, where the data is specific to the stage PS4. The computing device 102 is then configured to remove from consideration, at 110, the data for each hybrid that does not meet the threshold(s) of the product definition, per parameter. For example, the computing device 102 is configured to assess the car height of a hybrid, relative to a threshold of the product definition, and then, the lodging, and then the yield, etc., relative to respective thresholds. The hybrid may be removed from consideration when the specific threshold is not satisfied. Additionally, or alternatively, the computing device 102 may be configured with one or more rules, relative to the thresholds, whereby combinations of one or more satisfied thresholds may prevent a hybrid from being removed from consideration. For example, where a hybrid has a lodging just below the associated threshold, but the yield is relatively high, the hybrid may avoid being removed from consideration based on a rule. It should be appreciated that the one or more rules may be employed to account for overall priorities in the parameters, or to account for product knowledge of breeders associated with the breeding pipeline 106.

Once the considered hybrids are defined, the data for the considered hybrids is retrieved from the database 104, if not already done so, and the computing device 102 is configured to measure the parameters of the product definition in the data for the considered hybrids to define the prior stage thresholds. That is, for example, the computing device 102 is configured, at 112, to measure the parameters of the product definition in the BLUPs associated with the considered hybrids for each of the threshold. The computing device 102 is configured to then aggregate the measurements of the individual hybrids, for example, through a mean or average of the value per parameter. The aggregated measurements then define the prior stage thresholds based on the aggregate measurement. In one example, the prior stage thresholds are defined as one standard deviation below the mean measurement for the hybrids for the parameter. In this way, the thresholds are converted for the prior stages. For example, where the PS4 stage hybrids are considered, the product definition thresholds are converted to the prior stage threshold for the PS3 stage. In this example, the BLUPs may revert to the cohort mean with fewer data points, which may be typical in the example breeding pipeline. This may be instructive of choice of measure (e.g., one standard deviation in the above example, etc.) to apply in various embodiments. Additionally, or alternatively, the choice of measure may be informed by machine learning or other methods (e.g., with heritability influence, etc.) as appropriate or available.

Next, the computing device 102 is configured to proceed to the next iteration of the architecture 108, where the PS3 hybrid data is retrieved and assessed as described above, to convert the PS3 stage threshold into the PS2.5 stage threshold. The iterative operations from stage to stage then continue.

Additionally, as shown in the architecture 108, when the hybrids are considered (e.g., based on the calculated threshold(s), etc.), the computing device 102 is further configured, at 114, to identify the inbred parents of the considered hybrids. Like above, the computing device 102 is configured to retrieve the inbred data for the identified inbred parents from the considered hybrids of the target advancement stage (e.g., of the PS2.5 hybrids if the decision is to advance from PS2, etc.). The computing device 102 is configured, at 116, to measure the parameters of the product definition, and, the computing device 102 is configured to then aggregate the measurements of the individual inbreds, for example, by grouping females separately from males, or together, if desired, thresholds may then be applied equivalently to both parent groups. A measure of relevant parent groups follows similarly, as done for hybrids, although is not limited to be the same measure applied (e.g., is not limited to one standard deviation less of the mean, etc.). The aggregates of the measurements, as above, then, define the thresholds (for inbreds) for the product definition in the prior stage.

Next, as above, the computing device 102 is configured to proceed to the next iteration, where iterative operations, for the inbred threshold for the product concept are defined, from stage to stage.

It should be appreciated that, through the architecture 108, the product definition may be converted from PS4 to PS3, and from PS3, to PS2.5, and from PS2.5, to PS2, and from PS2 to PS1, and, from PS1, to SC2, etc., in this example embodiment. It should be appreciated that the conversion may include other stages, or more or less stages in other system embodiments. It should further be appreciated that the computing device 102 may be configured to repeat the above operation for each product definition, when multiple product definitions are being pursued at one or more times in the breeding pipeline 106.

Once the thresholds for the product definitions are defined, per stage, the computing device 102 is configured to impose the threshold for the specific stage on selection and/or advancement decisions for that stage. When multiple product definitions are in play for the breeding pipeline 106, at that stage, the respective thresholds may be combined. In doing so, the computing device 102 is configured to select the least restrictive threshold for each parameter across the different thresholds for the different product definitions to impose on the selection and/or advancement decisions. It should be appreciated that the thresholds may be combined otherwise, in total or per parameter in other embodiments.

In this manner, the computing device 102 is configured to make breeding decisions based on the converted thresholds of the product definition at one or more stages of the breeding pipeline 106, whereby some inbred and/or hybrids are advanced in the pipeline, while other ones of the inbreds and/or hybrids are removed from the pipeline.

It should be appreciated that the computing device 102 may be configured to display the one or more hybrids and/or inbreds that satisfy advancement thresholds and/or removal thresholds to the breeder, prior to removing the hybrid(s)/inbred(s) from the pipeline, along with parameter data for the hybrids/inbred. The breeder may then be able to assess the hybrid(s)/inbred(s) meeting the threshold conditions for removal from the pipeline. In one or more examples, then, the breeder may select one or more hybrids and/or inbreds to advance, despite the threshold indicating that the hybrid(s) inbred(s) should be removed from the pipeline, whereby the computing device 102 may be configured to accept an input from the breeder to override the removal threshold for one or more hybrids and/or inbreds.

In connection with the above, for the one or more hybrids and/or inbreds that are advanced in the pipeline, the computing device 102 may be configured to direct testing thereof, for example, wherein the hybrids and/or inbreds are planted (in growing spaces), grown, harvested, and tested as part of the pipeline. For instance, in one example, at least one plant is planted, consistent with the given inbred lines, in a field included in the breeding pipeline.

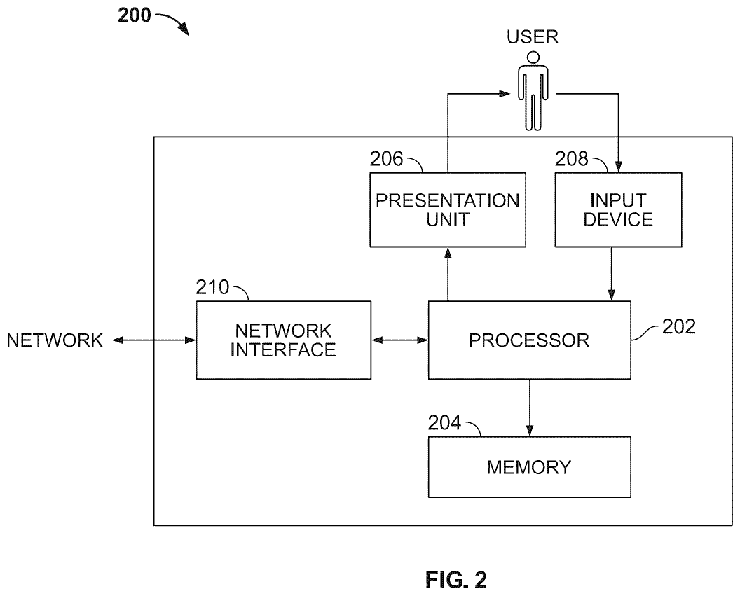

FIG. 2 illustrates an example computing device 200 that may be used in the system 100 of FIG. 1. The computing device 200 may include, for example, one or more servers, workstations, personal computers, laptops, tablets, smartphones, virtual devices/machines, etc. In addition, the computing device 200 may include a single computing device, or it may include multiple computing devices located in close proximity or distributed over a geographic region, so long as the computing devices are specifically configured to operate as described herein. In the example embodiment of FIG. 1, the computing device 102 includes and/or is implemented in one or more computing devices consistent with computing device 200. The database 104 may also be understood to include and/or be implemented in one or more computing devices, at least partially consistent with the computing device 200. However, the system 100 should not be considered to be limited to the computing device 200, as described below, as different computing devices and/or arrangements of computing devices may be used. In addition, different components and/or arrangements of components may be used in other computing devices.

As shown in FIG. 2, the example computing device 200 includes a processor 202 and a memory 204 coupled to (and in communication with) the processor 202. The processor 202 may include one or more processing units (e.g., in a multi-core configuration, etc.). For example, the processor 202 may include, without limitation, a central processing unit (CPU), a microcontroller, a reduced instruction set computer (RISC) processor, a graphics processing unit (GPU), an application specific integrated circuit (ASIC), a programmable logic device (PLD), a gate array, and/or any other circuit or processor capable of the functions described herein.

The memory 204, as described herein, is one or more devices that permit data, instructions, etc., to be stored therein and retrieved therefrom. In connection therewith, the memory 204 may include one or more computer-readable storage media, such as, without limitation, dynamic random access memory (DRAM), static random access memory (SRAM), read only memory (ROM), erasable programmable read only memory (EPROM), solid state devices, flash drives, CD-ROMs, thumb drives, floppy disks, tapes, hard disks, and/or any other type of volatile or nonvolatile physical or tangible computer-readable media for storing such data, instructions, etc. In particular herein, the memory 204 is configured to store data including, without limitation, parameter data (e.g., BLUPs, particular parameter values, etc.), product definitions, and/or other types of data (historical or otherwise) (and/or data structures) suitable for use as described herein.

Furthermore, in various embodiments, computer-executable instructions may be stored in the memory 204 for execution by the processor 202 to cause the processor 202 to perform one or more of the operations described herein (e.g., one or more of the operations of method 300, etc.) in connection with the various different parts of the system 100, such that the memory 204 is a physical, tangible, and non-transitory computer readable storage media. Such instructions often improve the efficiencies and/or performance of the processor 202 that is performing one or more of the various operations herein, whereby such performance may transform the computing device 200 into a special-purpose computing device. It should be appreciated that the memory 204 may include a variety of different memories, each implemented in connection with one or more of the functions or processes described herein.

In the example embodiment, the computing device 200 also includes an output device 206 that is coupled to (and is in communication with) the processor 202 (e.g., a presentation unit, etc.). The output device 206 may output information (e.g., thresholds, converted thresholds, product definitions, inbreds/hybrids removed from consideration, etc.), visually or otherwise, to a user of the computing device 200, such as a researcher, grower, technician, etc. It should be further appreciated that various interfaces (e.g., as defined by network-based applications, websites, etc.) may be displayed or otherwise output at computing device 200, and in particular at output device 206, to display, present, etc., certain information or data (as described herein) to the user. The output device 206 may include, without limitation, a liquid crystal display (LCD), a light-emitting diode (LED) display, an organic LED (OLED) display, an “electronic ink” display, speakers, a printer, etc. In some embodiments, the output device 206 may include multiple devices. Additionally, or alternatively, the output device 206 may include printing capability, enabling the computing device 200 to print text, images, and the like on paper and/or other similar media.

In addition, the computing device 200 includes an input device 208 that receives inputs from the user (i.e., user inputs) such as, for example, specify a product definition, override a removal of a hybrid/inbred from the breeding pipeline 106, etc. The input device 208 may include a single input device or multiple input devices. The input device 208 is coupled to (and is in communication with) the processor 202 and may include, for example, one or more of a keyboard, a pointing device, a touch sensitive panel, or other suitable user input devices. It should be appreciated that in at least one embodiment the input device 208 may be integrated and/or included with the output device 206 (e.g., a touchscreen display, etc.).

Further, the illustrated computing device 200 also includes a network interface 210 coupled to (and in communication with) the processor 202 and the memory 204. The network interface 210 may include, without limitation, a wired network adapter, a wireless network adapter, a mobile network adapter, or other device capable of communicating to one or more different networks (e.g., one or more of a local area network (LAN), a wide area network (WAN) (e.g., the Internet, etc.), a mobile network, a virtual network, and/or another suitable public and/or private network, etc.), including suitable networks capable of supporting wired and/or wireless communication between the computing device 200 and other computing devices, including with other computing devices used as described herein (e.g., between the computing device 102, the database 104, etc.).

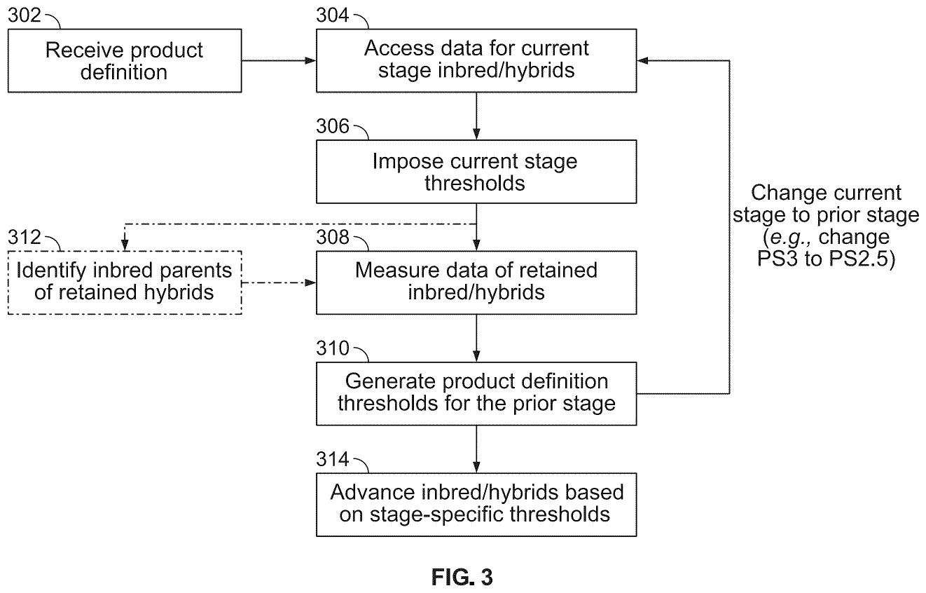

FIG. 3 illustrates an example method 300 for converting thresholds in plant breeding. The example method 300 is described herein in connection with the system 100, and may be implemented, in whole or in part, in the computing device 102 of the system 100. Further, for purposes of illustration, the example method 300 is also described with reference to the computing device 200 of FIG. 2. However, it should be appreciated that the method 300, or other methods described herein, are not limited to the system 100 or the computing device 200. And, conversely, the systems, data structures, and the computing devices described herein are not limited to the example method 300.

At the outset, it should be appreciated that method 300 is applied in connection with the breeding pipeline 106, for a specific plant (e.g., soybean, corn, etc.), which includes a defined sequence of stages, from SC2, PS1, PS2, PS2.5 (inbred to hybrid), PS3, PS4, and CM, in order to develop a commercial product. It should be appreciated that multiple products may be developed at one time, whereby various different product definitions may be imposed on the breeding pipeline 106 at one time, whether the product definitions were imposed at the same time, or at different times.

In connection therewith, at 302, the computing device 102 receives one or more product definitions, for example, from the breeder in charge of and/or associated with the breeding pipeline 106. In this example, the one or more product definitions includes, for instance, and without limitation, three thresholds for three specific parameters (e.g., yield, lodging, test weight, etc.). For purposes of illustration here, only one product definition is referenced below. That said, it should be understood, for multiple product definitions, the method steps below are generally duplicated.

At 304, the computing device 102 accesses data for a current stage of the iterative process (e.g., in architecture 108, etc.). In this example, the method 300 begins with stage PS4. As such, the historical data for hybrids in stage PS4 is accessed. The data is accessed from the database 104, and may include data for all hybrids in the PS4 stage of the breeding pipeline 106, over the prior one or more years.

At 306, the computing device 102 imposes the thresholds of the product definition to the accessed data, per parameter, as a removal/advance threshold with regard to subsequent stages. The thresholds may be imposed strictly, or may be imposed in combination with one or more deviations, or may be imposed in combination with one or more rules. One example rule may be used, in combination with the thresholds, whereby a specific hybrid may fail to satisfy the test weight threshold itself, but may satisfy the test weight threshold when one or more standard deviations is applied (e.g., one standard deviation in the worse performance direction of the mean for the test weight across the hybrids, etc.). In this way, the thresholds, per parameter, may be imposed, yet allowance may be provided for certain desired parameters (e.g., yield, etc.) that do not strictly satisfy the thresholds.

It should be appreciated that one or more different rules may be applied to assist, augment, or otherwise aid the thresholds, per parameter, in removing or considering certain of the hybrids, as desired or required, with regard to advancement. It should be appreciated that one or more rules may be applied in one iteration of method 300, while the same, or other, or different rules may be applied in other iterations. It should also be appreciated, again, that the hybrids removed, per stage, in this example, are not necessarily dropped from the pipeline 106. They may remain in the pipeline 106, but not advanced to next stages in this example.

As indicated, the computing device 102 imposes the removal/consideration threshold, per parameter, whereby the computing device 102, in this example, assesses each hybrid three times, one for each of the three thresholds of the product definition.

Thereafter, for the considered (or retained) hybrids, the computing device 102 measures, at 308, the parameters (e.g., yield, lodging, test weight, etc.) of the hybrids. The measuring may be directed to BLUP data for the hybrids, which is indicative of one or more of the parameters. For example, yield of a hybrid/inbred may be expressed as a BLUP, which is a linear regression or adjusted mean of the yield data based on the historical data for the hybrid/inbred (e.g., over one year, two years, or three years, etc.). It should be appreciated that the data in the database 104 may include BLUPs for one or various parameters of each of the hybrids/inbreds included in the database 104 for one or more of the same or different intervals. For example, the database 104 may include individual BLUPs, per hybrid/inbred (for one or more intervals (e.g., year, multiple years, etc.), etc.), for a three year interval, for example: car height (EHT), green snap percentage (GSPP), moisture best estimation (MST_BE), plant height (PHT), root lodging percentage (RTLP), selection index (SLIN), stalk lodging percentage (STLP), total test weight (TWT), yield best estimation (YLD_BE), Goss's Wilt (GW) resistance rating, Southern Rust (SR) resistance rating, Gray Leaf Spot, Northern Corn Leaf Blight resistance (or resistance rating), Diplodia Ear Rot resistance (or resistance rating), Fusarium Ear Rot resistance (or resistance rating), Gibberella Ear Rot resistance (or resistance rating), Anthracnose Stalk Rot resistance (or resistance rating), etc. It should be appreciated that more or less, or different, parameters may be represented by BLUPs, simulations, or otherwise, in other system embodiments. It should further be appreciated that the computing device 102 may measure data other than BLUP data for one or more of the parameters of the product definition, based on, for example, the availability of data, the type of parameters, etc.

The computing device 102 then generates, at 310, product definition thresholds for the prior stage, which in this example, is stage PS3. The threshold, per parameter, is generated based on an aggregate of the measures from data for the parameter, which may include an average, mean, or other suitable combination, either in combination, or not, with one or more standard deviations, absolute divisions, or other suitable statical technique, etc.

As shown in FIG. 3, the method 300 then continues to step 304, whereupon the current stage is changed to the prior stage (i.e., the conversion works backwards in the stages). In this example, as this point, the current stage is changed from PS4 to PS3. Consequently, the method 300 continues at step 304 to access historical data for the hybrids from stage PS3 consistent with the description above.

As further shown in FIG. 3, the method optionally (as indicated by the dotted lines) includes, for the hybrid stages, the branch in which the computing device 102 identifies, at 312, the inbred parents of the considered/retained hybrids (after imposing the threshold, at step 306) and, optionally, again imposes the threshold on the inbred parents of the considered/retained hybrids. The method then continues, at 308, where the computing device 102 measures the parameter data for the accessed data for the inbreds. While optional in connection with the hybrid stages, i.e., PS4, PS3, etc., the identification of the inbred parent is necessary when changing from stage PS2.5 to stage PS2, which is an inbred stage. The inbreds are then assessed to further convert the product definition to a threshold appropriate for the specific stage.

It should be understood that the method 300 may be repeated, or iterated, for multiple different product definitions. The method 300 may be iterated for each stage of the plant breeding pipeline 106, or only a portion of the stages. For example, where a breeding decision is being made to select an inbred in stage PS2, there may be limited or no reason to convert the product definition threshold backwards to PS1 or SC2.

At 314, the computing device 102 advances one or more inbred/hybrids in the breeding pipeline 106, in a specific stage thereof, based on one or more converted stage-specific threshold for the stage. The advancement may be automated, by the computing device 102, or based on approval, assessed, or reviewed by a breeder associated with the breeding pipeline 106 and decisions related to the stage. In addition, while not shown, the product definition threshold, per stage, per parameter, may be combined between different product definitions to make selection decisions across different product definitions, as referenced above, so that early stage inbred/hybrids may potentially advance into products consistent with any one or more of the product definitions. In connection with such advancement in the breeding pipeline 106, in some examples, the inbred/hybrids may be planted in a growing space (e.g., a field, a greenhouse, etc.) and tested. For instance, with reference to FIG. 1, the selected inbred/hybrids may be advanced to a breeding stage PS2.5 of the breeding pipeline 106, planted in a field during the next growing season, and tested (in field) as part of PS2.5 products.

In view of the above, the systems and methods herein provide for converting thresholds in plant breeding. In general, a product definition is provided for a hybrid product to be commercialized. Because the breeding pipeline 106, by which the hybrid is development is intended to gain in performance from stage to stage, generally, the product definition may be too stringent, or too aggressive for earlier stages, especially as the stages get further away from the CM stage, for example. When applied to the earlier stages, therefore, inbred/hybrids may be unnecessarily, or mistakenly, removed from consideration from the breeding pipeline 106. By converting the product definition, and specifically, the per parameter threshold therein, to sensible levels for the specific stage of the breeding pipeline 106, based on specific analysis of the underlying data of detained inbred/hybrids, the computing device provides for enhanced decision making, efficiencies and broader pool of inbred/hybrids to achieve the product definition.

With that said, it should be appreciated that the functions described herein, in some embodiments, may be described in computer executable instructions stored on a computer readable media, and executable by one or more processors. The computer readable media is a non-transitory computer readable media. By way of example, and not limitation, such computer readable media can include RAM, ROM, EEPROM, CD-ROM or other optical disk storage, magnetic disk storage or other magnetic storage device, or any other medium that can be used to carry or store desired program code in the form of instructions or data structures and that can be accessed by a computer. Combinations of the above should also be included within the scope of computer-readable media.

It should also be appreciated that one or more aspects of the present disclosure may transform a general-purpose computing device into a special-purpose computing device when configured to perform one or more of the functions, methods, and/or processes described herein.

As will be appreciated based on the foregoing specification, the above-described embodiments of the disclosure may be implemented using computer programming or engineering techniques, including computer software, firmware, hardware or any combination or subset thereof, wherein the technical effect may be achieved by performing at least one of the following operations: (a) accessing data indicative of multiple hybrid plants at a first stage of a breeding pipeline, the data including values for one or more parameters of the multiple hybrid plants; (b) imposing, on the accessed data, one or more first thresholds included in a product definition for the breeding pipeline, whereby ones of the multiple hybrid plants are considered at the first stage of the breeding pipeline based on satisfying the one or more first thresholds, each of the one or more first thresholds specific to one of the one or more parameters; (c) measuring the values of the one or more parameters for the considered ones of the multiple hybrid plants; (d) generating, based on an aggregate of the measured values of the one or more parameters for the considered ones of the multiple hybrid plants, one or more second thresholds; € repeating steps (a)-(d) for a second stage of the breeding pipeline with the one or more second thresholds in place of the one or more first thresholds; (f) receiving the product definition from a breeder associated with the breeding pipeline; (g) identifying inbred plants as parents of each of the considered ones of the multiple hybrid plants, and then: (i) accessing data indicative of the multiple inbred plants at a third stage of the breeding pipeline, the data including values for the one or more parameters of the inbred plants; (ii) imposing, on the accessed data indicative of the inbred plants, one or more third thresholds, whereby ones of the inbred plants are considered at the third stage of the breeding pipeline based on satisfying the one or more third thresholds, each of the one or more third thresholds specific to one of the one or more parameters of the inbred plants; (iii) measuring the values of the one or more parameters of the inbred plants for the considered ones of the inbred plants; (iv) generating, based on an aggregate of the measured values of the one or more parameters for the considered ones of the inbred plants, one or more fourth thresholds; and (v) repeating steps (i)-(iv) for a fourth stage of the breeding pipeline with the one or more fourth thresholds in place of the one or more third thresholds; (h) advancing, from the second stage of the breeding pipeline, one or more of the multiple hybrid plants based on the second thresholds; and/or (i) advancing, from the fourth stage of the breeding pipeline, the one or more of the multiple inbred plants based on the fourth thresholds.

Examples and embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail. In addition, advantages and improvements that may be achieved with one or more example embodiments disclosed herein may provide all or none of the above-mentioned advantages and improvements and still fall within the scope of the present disclosure.

Specific values disclosed herein are example in nature and do not limit the scope of the present disclosure. The disclosure herein of particular values and particular ranges of values for given parameters are not exclusive of other values and ranges of values that may be useful in one or more of the examples disclosed herein. Moreover, it is envisioned that any two particular values for a specific parameter stated herein may define the endpoints of a range of values that may also be suitable for the given parameter (i.e., the disclosure of a first value and a second value for a given parameter can be interpreted as disclosing that any value between the first and second values could also be employed for the given parameter). For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, −1-2, 2-10, 2-8, 2-3, 3-10, and 3-9.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When a feature is referred to as being “on,” “engaged to,” “connected to,” “coupled to,” “associated with,” “in communication with,” or “included with” another element or layer, it may be directly on, engaged, connected or coupled to, or associated or in communication or included with the other feature, or intervening features may be present. As used herein, the term “and/or” and the phrase “at least one of” includes any and all combinations of one or more of the associated listed items.

Although the terms first, second, third, etc. may be used herein to describe various features, these features should not be limited by these terms. These terms may be only used to distinguish one feature from another. Terms such as “first,” “second,” and other numerical terms when used herein do not imply a sequence or order unless clearly indicated by the context. Thus, a first feature discussed herein could be termed a second feature without departing from the teachings of the example embodiments.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A computer-implemented method for use in converting thresholds in plant breeding, the method comprising:
(a) accessing, by a computing device, data indicative of multiple hybrid plants at a first stage of a breeding pipeline, the data including values for one or more parameters of the multiple hybrid plants;
(b) imposing, by the computing device, on the accessed data, one or more first thresholds included in a product definition for the breeding pipeline, whereby ones of the multiple hybrid plants are considered at the first stage of the breeding pipeline based on satisfying the one or more first thresholds, each of the one or more first thresholds specific to one of the one or more parameters;
(c) measuring the values of the one or more parameters for the considered ones of the multiple hybrid plants;
(d) generating, based on an aggregate of the measured values of the one or more parameters for the considered ones of the multiple hybrid plants, one or more second thresholds;
repeating steps (a)-(d) for a second stage of the breeding pipeline with the one or more second thresholds in place of the one or more first thresholds; and
advancing one or more prior hybrid plants from the second stage to the first stage based on the second thresholds.

2. The computer-implemented method of claim 1, further comprising receiving, by the computing device, the product definition from a breeder associated with the breeding pipeline, prior to accessing the data indicative of the multiple hybrid plants.

3. The computer-implemented method of claim 1, wherein the one or more parameters include yield, test weight, lodging, stature, Goss's wilt resistance, southern rust resistance, gray leaf spot resistance, northern corn leaf blight resistance, and/or anthracnose stalk rot resistance.

4. The computer-implemented method of claim 3, wherein the one or more parameters include: yield, test weight, lodging, and/or stature.

5. The computer-implemented method of claim 1, wherein (c) measuring the values of the one or more parameters includes (c) measuring best linear unbiased prediction (BLUP) values for the one or more parameters.

6. The computer-implemented method of claim 1, further comprising identifying inbred plants as parents of each of the considered ones of the multiple hybrid plants; and then
(e) accessing, by the computing device, data indicative of the multiple inbred plants at a third stage of the breeding pipeline, the data including values for the one or more parameters of the inbred plants;
(f) imposing, by the computing device, on the accessed data indicative of the inbred plants, one or more third thresholds, whereby ones of the inbred plants are considered at the third stage of the breeding pipeline based on satisfying the one or more third thresholds, each of the one or more third thresholds specific to one of the one or more parameters of the inbred plants;
(g) measuring the values of the one or more parameters of the inbred plants for the considered ones of the inbred plants;
(h) generating, based on an aggregate of the measured values of the one or more parameters for the considered ones of the inbred plants, one or more fourth thresholds; and
repeating steps (e)-(h) for a fourth stage of the breeding pipeline with the one or more fourth thresholds in place of the one or more third thresholds.

7. The computer-implemented method of claim 6, further comprising:
advancing, from the second stage of the breeding pipeline, one or more of the multiple hybrid plants based on the one or more second thresholds; and
advancing, from the fourth stage of the breeding pipeline, the one or more of the multiple inbred plants based on the one or more fourth thresholds.

8. The computer-implemented method of claim 7, further comprising advancing, from the second stage of the breeding pipeline, one or more of the multiple hybrid plants based on the one or more second thresholds;
wherein advancing the one or more of the multiple hybrid plants includes:
automatically directing the one or more of the multiple hybrid plants from the second stage of the breeding pipeline to a third stage of the breeding pipeline in response to the one or more of the multiple hybrid plants satisfying the one or more second thresholds; and/or
automatically planting at least one plant, consistent with the one or more of the multiple hybrid plants, in a field included in the breeding pipeline in response to the one or more of the multiple hybrid plants satisfying the one or more second thresholds.

9. A non-transitory computer-readable storage medium including executable instructions, which when executed by at least one processor, cause the at least one processor to:
(a) access data indicative of multiple hybrid plants at a first stage of a breeding pipeline, the data including values for one or more parameters of the multiple hybrid plants;
(b) impose, on the accessed data, one or more first thresholds included in a product definition for the breeding pipeline, whereby ones of the multiple hybrid plants are considered at the first stage of the breeding pipeline based on satisfying the one or more first thresholds, each of the one or more first thresholds specific to one of the one or more parameters;
(c) determine the values of the one or more parameters for the considered ones of the multiple hybrid plants;
(d) generate, based on an aggregate of the determined values of the one or more parameters for the considered ones of the multiple hybrid plants, one or more second thresholds;
repeat steps (a)-(d) for a second stage of the breeding pipeline with the one or more second thresholds in place of the one or more first thresholds; and
direct one or more prior hybrid plants from the second stage to the first stage based on the second thresholds.

10. The non-transitory computer-readable storage medium of claim 9, wherein the executable instructions, when executed by the at least one processor, further cause the at least one processor to receive the product definition from a breeder associated with the breeding pipeline, prior to accessing the data indicative of the multiple hybrid plants.

11. The non-transitory computer-readable storage medium of claim 9, wherein the one or more parameters include: yield, test weight, lodging, and/or stature.

12. The non-transitory computer-readable storage medium of claim 9, wherein the executable instructions, when executed by the at least one processor to (c) determine the values of the one or more parameters, cause the at least one processor to (c) measure best linear unbiased prediction (BLUP) values for the one or more parameters.

13. The non-transitory computer-readable storage medium of claim 9, wherein the executable instructions, when executed by the at least one processor, further cause the at least one processor to identify inbred plants as parents of each of the considered ones of the multiple hybrid plants; and then
(e) access data indicative of the multiple inbred plants at a third stage of the breeding pipeline, the data including values for the one or more parameters of the inbred plants;
(f) impose, on the accessed data indicative of the inbred plants, one or more third thresholds, whereby ones of the inbred plants are considered at the third stage of the breeding pipeline based on satisfying the one or more third thresholds, each of the one or more third thresholds specific to one of the one or more parameters of the inbred plants;
(g) determine the values of the one or more parameters of the inbred plants for the considered ones of the inbred plants;
(h) generate, based on an aggregate of the measured values of the one or more parameters for the considered ones of the inbred plants, one or more fourth thresholds; and
repeat steps (e)-(h) for a fourth stage of the breeding pipeline with the one or more fourth thresholds in place of the one or more third thresholds.

14. A system for use in converting thresholds in plant breeding, the system comprising at least one computing device configured to:
(a) access data indicative of multiple hybrid plants at a first stage of a breeding pipeline, the data including values for one or more parameters of the multiple hybrid plants;
(b) impose, on the accessed data, one or more first thresholds included in a product definition for the breeding pipeline, whereby ones of the multiple hybrid plants are considered at the first stage of the breeding pipeline based on satisfying the one or more first thresholds, each of the one or more first thresholds specific to one of the one or more parameters;
(c) measure the values of the one or more parameters for the considered ones of the multiple hybrid plants;
(d) generate, based on an aggregate of the measured values of the one or more parameters for the considered ones of the multiple hybrid plants, one or more second thresholds;
repeat steps (a)-(d) for a second stage of the breeding pipeline with the one or more second thresholds in place of the one or more first thresholds; and
direct advancement of one or more prior hybrid plants from the second stage to the first stage based on the second thresholds.

15. The system of claim 14, wherein the at least one computing device is further configured to receive the product definition from a breeder associated with the breeding pipeline, prior to accessing the data indicative of the multiple hybrid plants.

16. The system of claim 14, wherein the one or more parameters include: yield, test weight, lodging, and/or stature.

17. The system of claim 14, wherein the at least one computing device is configured, in order to (c) determine the values of the one or more parameters, to (c) measure best linear unbiased prediction (BLUP) values for the one or more parameters.

18. The system of claim 14, wherein the at least one computing device is further configured to identify inbred plants as parents of each of the considered ones of the multiple hybrid plants; and then
(e) access data indicative of the multiple inbred plants at a third stage of the breeding pipeline, the data including values for the one or more parameters of the inbred plants;
(f) impose, on the accessed data indicative of the inbred plants, one or more third thresholds, whereby ones of the inbred plants are considered at the third stage of the breeding pipeline based on satisfying the one or more third thresholds, each of the one or more third thresholds specific to one of the one or more parameters of the inbred plants;
(g) determine the values of the one or more parameters of the inbred plants for the considered ones of the inbred plants;
(h) generate, based on an aggregate of the measured values of the one or more parameters for the considered ones of the inbred plants, one or more fourth thresholds; and
repeat steps (e)-(h) for a fourth stage of the breeding pipeline with the one or more fourth thresholds in place of the one or more third thresholds.
